(19) United States
(12) Patent Application Publication
(10) Pub. No.: US 2021/0386762 A1
(43) Pub. Date: Dec. 16, 2021

(54) THE APPLICATION OF URSODEOXYCHOLIC ACID OR ITS PHARMACEUTICAL SALT IN THE PREPARATION OF AN ANTITUMOR DRUG AND AN ANTITUMOR DRUG

(71) Applicant: ZHEJIANG UNIVERSITY, HANGZHOU, ZHEJIANG PROVINCE (CN)

(72) Inventors: ZHIJIAN CAI, HANGZHOU, ZHEJIANG PROVINCE (CN); JIANLI WANG, HANGZHOU, ZHEJIANG PROVINCE (CN); YINGYING SHEN, HANGZHOU, ZHEJIANG PROVINCE (CN); CHAOJIE LU, HANGZHOU, ZHEJIANG PROVINCE (CN); ZHENGBO SONG, HANGZHOU, ZHEJIANG PROVINCE (CN)

(21) Appl. No.: 17/286,442

(22) PCT Filed: Sep. 9, 2019
(86) PCT No.: PCT/CN2019/104831

§ 371 Date: Apr. 16, 2021

(30) Foreign Application Priority Data
Nov. 21, 2018 (CN) 201811391474.8

(51) Int. Cl. A61K 31/575; A61K 39/395; A61P 35/00

(52) U.S. Cl. CPC A61K 31/575; A61P 35/00; A61K 39/3955

(57) ABSTRACT

The present invention discloses the application of an ursodeoxycholic acid and its pharmaceutical salt in the preparation of an antitumor drug and an antitumor drug. Through research, it is discovered that ursodeoxycholic acid and its pharmaceutical salt have good inhibition effect on a variety of tumor cells, and further research reveals that they function by reducing the proportion of regulatory T cells in tumor infiltrating lymphocytes. Therefore, ursodeoxycholic acid can be used in the preparation of an antitumor drug. Besides, as a clinically used choleretic drug, ursodeoxycholic acid does not require clinical safety evaluation, and has good application prospects. It can also be used as an ancillary drug to improve the therapeutic effect of anti-PD-1 drugs.

### TECHNICAL FIELD

The present invention relates to the field of biomedical technology. More particularly, the present invention relates to the application of ursodeoxycholic acid or its pharmaceutical salt in the preparation of an antitumor drug and an antitumor drug.

### BACKGROUND TECHNOLOGY

With the year-by-year increase of tumor incidence rate and mortality rate, malignant tumor has become a major chronic disease seriously threatening human health, and one of the most severe public health issues in China and even the world. Governments around the world has shifted the focus of health strategy to tumor control. For patients at early and intermediate stages, surgery is the first choice for treatment. However, due to the insidious onset of tumor and difficulties in early diagnosis, most patients are diagnosed with locally advanced or remotely metastatic tumor at first, for which the main treatment methods are radiotherapy, chemotherapy and targeted therapy at present. Nevertheless, these treatment methods cannot bring long-term survival benefits to patients with advanced solid tumor. With the continuous development and integration of relevant disciplines including oncology, immunology and molecular biology, the fundamental and clinical researches of tumor immunotherapy have achieved rapid development, especially for immune checkpoint inhibitors. For instance, programmed death 1 (PD-1) and programmed death-ligand 1 (PD-L1) antibodies have brought long-term survival benefits to patients of various solid tumors such as advanced melanoma and nonsmall-cell lung cancer. In 2018, the Nobel Prize for Physiology or Medicine was awarded to Professor James P. Allison and Professor Tasuku Honjo, to commend their contributors to human tumor immunotherapy.

Despite the remarkable effect of immune checkpoint inhibitors in improving the survival rate of patients, only 10%-30% of solid tumor patients benefited from monotherapy. This is because, the immunosuppressive microenvironment within tumor is an important factor of ineffective antitumor immunity, where the infiltration of suppressor cells plays an indispensable role. Common suppressor cells include myeloid derived suppressor cells (MDSC), tumor-associated macrophages (TAM) and regulatory T cells (Treg), among which Treg-mediated immunosuppression is a major obstacle to effective tumor treatment. The infiltration of a large amount of Treg cells in the tumor tissues of patients with non-small cell lung cancer and ovarian cancer was first reported by Woo, et al. (Woo E Y, Chu C S, Goletz T J, et al. Regulatory CD4+CD25+ T cell in tumors from patients with early-stage non-small cell lung cancer and late-stage ovarian cancer. Cancer Res, 2001, 61:4766-72). Later, a series of reports indicated the aggregation of Treg cells in the paraneoplastic and carcinoma tissues of multiple tumors, including melanoma, breast cancer, colorectal cancer, lung cancer and pancreatic cancer (Zou W. Regulatory T cells, tumor immunity and immunotherapy. Nat Rev Immunol, 2006, 6:295-307). Many experiments showed that the increase Treg cells was conducive to the immune escape of tumors. In clinical cases, the increase of Treg cells in the microenvironment of tumors including breast cancer, ovarian cancer, gastric cancer and liver cancer was correlated with unfavorable prognosis. In terms of mechanism, Treg cells are capable of inhibiting extensive antitumor immune response while promoting angiogenesis in the tumor microenvironment. Therefore, selective removal or inhibition of Treg cells in the tumor microenvironment has become a new direction of tumor immunotherapy.

As a natural bile salt in human bile, ursodeoxychilic acid (UDCA) is produced by the reduction of secondary bile acid in the intestinal tract, and functions to sooth the gallbladder and protect liver cells. Clinically it is often used to prevent and treat cholecystitis, cholangitis, biliary dyspepsia, and jaundice caused by cholesterol calculus or calculus. A recent study showed UDCA could inhibit the participation of cell factors including TNF-α, IL-1β and IL6 in the regulation of inflammatory reaction (Ko, W K., Kim, S. J., Jo, M J. et al. Mol Neurobiol 2018).

### SUMMARY OF INVENTION

The present invention verifies through research that ursodeoxychilic acid (UDCA) has antitumor effect, and can be used in the preparation of an antitumor drug.

The chemical formula of UDCA is demonstrated as Formula I:

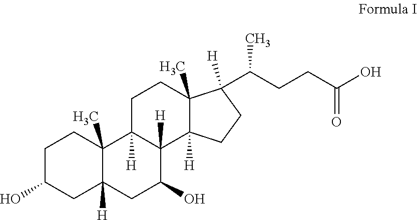

The present invention for the first time provides the application of ursodeoxycholic acid or its pharmaceutical salt in the preparation of an antitumor drug.

The ursodeoxycholic acid or its pharmaceutical salt functions by reducing the proportion of Treg cells in tumor infiltrating lymphocytes.

The ursodeoxycholic acid or its pharmaceutical salt is used in combination with an anti-PD-1 drug.

The types of tumor targeted by the antitumor drug include melanoma, rectal cancer and lung cancer.

The present invention also provides an antitumor drug, which includes ursodeoxycholic acid or its pharmaceutical salt.

The antitumor drug also includes an anti-PD-1 drug.

Preferably, the anti-PD-1 drug is an anti-PD-1 monoclonal drug. For example, the SHR-1210 produced by Hengrui Medicine is a humanized anti-PD-1 antibody.

The types of tumor targeted by the antitumor drug include melanoma, rectal cancer and lung cancer.

Through in vitro experiments, the present invention finds that ursodeoxycholic acid inhibits endogenous Treg cell differentiation. To explore the effect of this drug in human body, it is found via three tumor models (melanoma, rectal cancer and lung cancer) that ursodeoxycholic acid markedly inhibits tumor growth in a tumor-bearing model. Further analysis of tumor infiltrating lymphocytes with flow cytometry reveals: in the tumor-bearing model, the proportion of Treg cells in tumor infiltrating lymphocytes is obviously reduced in the group treated with ursodeoxycholic acid. Through combined treatment with ursodeoxycholic acid and anti-PD-1 antibodies, it is discovered that ursodeoxycholic acid can greatly enhance the therapeutic effect of anti-PD-1 antibodies.

This research in the present invention found out that ursodeoxycholic acid or its pharmaceutical salt has good inhibition effect on a variety of tumor cells, and can be used in the preparation of an anti-tumor drug. As a clinically used choleretic drug, ursodeoxycholic acid does not require clinical safety evaluation, and has good application prospects. It can also be used as an ancillary drug to improve the therapeutic effect of anti-PD-1 drugs.

### BRIEF DESCRIPTION OF DRAWINGS

FIG. 1 shows the test results of the influence of ursodeoxycholic acid on endogenous Treg cell differentiation, with the results of flow cytometry and statistics demonstrated in A and B respectively (*** represents P<0.001, and the same below).

FIGS. 2A, 2B and 2C display the test results of the antitumor effect of ursodeoxycholic acid in the melanoma mouse model. Specifically, FIG. 2A demonstrates the influence of ursodeoxycholic acid on tumor volume changes, FIG. 2B shows changes in the proportion of Treg cells in tumor infiltrating lymphocytes which are detected by flow cytometry, and FIG. 2C represents the statistical results (** represents P<0.01, and the same below).

FIG. 3A, FIGS. 3B and 3C demonstrate the antitumor effect of ursodeoxycholic acid as detected in the lung cancer mouse model. To be more specific, FIG. 3A reveals the influence of ursodeoxycholic acid on tumor volume changes, FIG. 3B shows the flow cytometry detection result of changes in the proportion of Treg cells in tumor infiltrating lymphocytes, and FIG. 3C reflects the statistical results (* represents P<0.05, and the same below).

FIGS. 4A, 4B and 4C exhibit the antitumor effect of ursodeoxycholic acid detected in the colon cancer mouse model. In particular, FIG. 4A shows the influence of ursodeoxycholic acid on tumor volume changes, FIG. 4B displays changes in the proportion of Treg cells in tumor infiltrating lymphocyte as detected by flow cytometry, and FIG. 4C demonstrates the statistical results.

FIG. 5 reveals how the combined use of ursodeoxycholic acid and humanized programmed death 1 (PD-1) affects tumor volume changes in the colon cancer mouse model.

FIG. 6 demonstrates how the combined use of ursodeoxycholic acid and humanized programmed death 1 (PD-1) affects tumor volume changes in the lung cancer mouse model.

FIG. 7 shows the influence of the combined use of ursodeoxycholic acid and humanized programmed death 1 (PD-1) on tumor volume changes in the melanoma mouse model.

FIG. 8 displays the detection results of how ursodeoxycholic acid reduces the differentiation of endogenous Treg cells, among which A demonstrates the flow cytometry detection results, and B represents the statistical results (* represents P<0.05, and *** represents P<0.001).

### SPECIFIC EMBODIMENTS OF THE PRESENT INVENTION

Female C57BL/6 (6-8 weeks old) mice were purchased from Shanghai SLAC Laboratory Animal Co., Ltd.; humanized PD-1 transgenic mice were purchased from Nanjing Biomedical Research Institute of Nanjing University; and all mice were raised in SPF facilities. Mouse colon cancer cell line MC38, lung cancer cell line LLC-luci and melanoma cell line B16 were all purchased from American Type Culture Collection (ATCC).

### Example 1

Treg cell surface can express CD4 and CD25 molecules. In addition, it is characterized by its high expression of transcription factor Foxp3. Based on the characteristic above, flow cytometry is adopted to explore the in vitro influence of ursodeoxycholic acid on Treg cell differentiation. Detailed procedures are as follows:

T cell sorting: Mouse T cells were sorted by immunomagnetic beads methods. The mice were killed by cervical dislocation, spleens and lymph nodes were taken under aseptic conditions, ground and put through a sieve, centrifuged at 400 g for 5 min, and cells were resuspended with sort buffer. The total cells were counted with 3% glacial acetic acid, and proceeded strictly following the instructions of the sorting kit.

CD4 negative selection kit (EasySep™ Mouse CD4+ T Cell Isolation Kit, #19852A) and Biotin sorting kit (EasySep™ Mouse Biotin Positive Selection Kit, #18556) were used in combination for sorting naïve CD4+ T cells (CD4-positive naïve T cells). First CD4+ T cells were sorted through negative selection, and then CD62L+ T cells were sorted out through positive selection. After sorting, when the CD4+CD62L+ purity detected by flow cytometry is greater than 95%, cells were used for further experiments.

In a 96-well plate, anti-CD3 and anti-CD28 (BioXcell, #145-2C11) were diluted to a concentration of 2 μg/ml with autoclaved PBS, the plate was wrapped at 200 μl/well, and stood for over 2 hours at 37° C. The naïve CD4+ T cells were sorted above into the 96-well plate at 4×10⁵ cells/well. DMSO or ursodeoxycholic acid U5127 (SIGMA-ALDRICH, #128-13-2) was added with a concentration of 50 μM respectively, and the differentiation of Treg cells were detected by flow cytometry on day 4. Detailed procedures were as follows:
(1) cells were collected to test tube, and rinsed with PBS solution.
(2) 0.2 μl of anti-mouse CD4 FITC (Biolegend, #100406) and anti-mouse CD25 APC (Biolegend, #101909) flow antibodies were added, and stained for 20 minutes under room temperature away from light.
(3) Rinsed with PBS solution once, perforated the nuclear membranes and fixate with 1 ml of eBioscience™ Foxp3/Transcription Factor Fixation/Permeabilization Concentrate and Diluent (Invitrogen, #00-5521-00) for 1 hour.
(4) Neutralized with 1× eBioscience™ Permeabilization Buffer (Invitrogen, #00-8333-56), and discarded the culture supernatant after centrifugalization.
(5) Added 0.5 μl of anti-mouse Foxp3 PE (eBioscience, #12-5773-82) flow antibodies, and stained at 4° C. for 1 hour.
(6) Neutralized with 1× Permeabilization Buffer, rinsed twice, discarded the culture supernatant, resuspended cells with an appropriate amount of PBS solution, and detected Treg cell differentiation by flow cytometry.

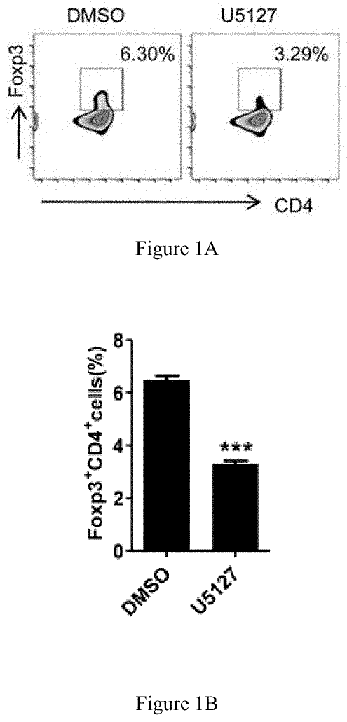

The result showed: the proportion of endogenous Treg cells was significantly reduced by ursodeoxycholic acid (FIG. 1).

### Example 2

Since Treg cells can inhibit extensive antitumor immune response while promoting angiogenesis in the tumor microenvironment, to explore the in vivo influence of ursodeoxycholic acid on Treg cells, tumor-bearing models are established using mouse colon cancer cell line MC38, lung cancer cell line LLC-luci and melanoma cell line B16, with female SPF-grade C57BL/6 as the experiment subject. For better internal absorption of ursodeoxycholic acid, the above-mentioned ursodeoxycholic acid is replaced with sodium ursodeoxycholate (SANTA CRUZ BIOTECHNOLOGY, #2898-95-5).

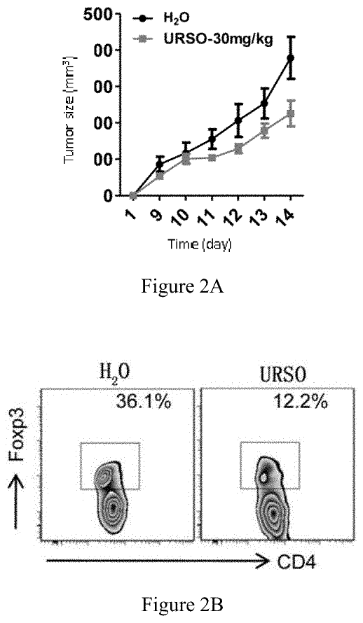

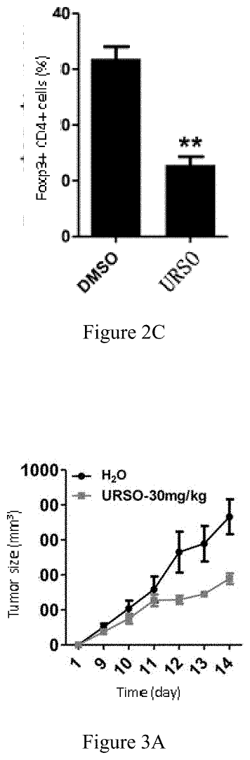

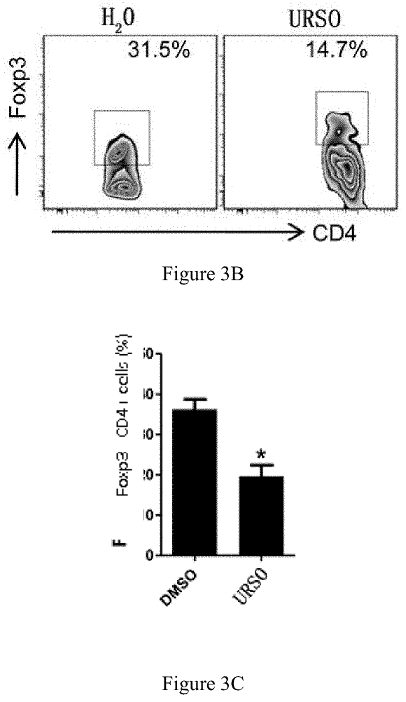

The control group and the experimental group both received injection of ddH₂O and URSO (30 mg/kg) every day, with tumor size observed and recorded. As shown in FIG. 2A, FIG. 3A and FIG. 4A, compared to the control group, the tumor-bearing mice in the experimental group are obviously smaller in volume.

Two weeks later, separate the tumor-infiltrating lymphocytes for analysis by flow cytometry. Detailed procedures are as follows:
(1) tumor tissues were placed in 1 ml of 1640 medium containing 10% serum, and chopped.
(2) Transferred to 10 ml Eppendorf tube and supplement medium until reaching 5 ml.
(3) Type IV collagenase 2 mg/ml (Sangon Biotech, #A004186) and deoxyribonuclease I 20 μg/ml (SIGMA-ALDRICH, #10104159001) were added, digested at 37° C. for 2 hours.
(4) After completion of digestion, the product of step (3) was filtered with 200 mesh nylon filter screen to obtain single-cell suspension, and discarded the culture supernatant after 4-5 minutes of centrifugal sedimentation at 1500 rpm at 4° C.
(4) Percoll (GE, #17-0819-09) separation medium was prepared with concentration at 1.082 g/ml, 1.075 g/ml, 1.07 g/ml and 1.06 g/ml, resuspend cells with high-density Percoll solution, added to 15 ml centrifuge tube in the order from the lowest to the highest density, centrifuged at 1500 rpm for 20 minutes at 4° C.
(5) Cell stratification was observed, cells were taken from the intermediate tunica albuginea layer, neutralized with PBS of twice the volume, centrifuged at 1500 rpm for 5 minutes at 4° C., and discarded the culture supernatant.
(6) Cells were transferred to test tube, and proceeded with the cellular staining procedures in Example 1.

According to the flow cytometry results (FIG. 2B, FIG. 3B and FIG. 4B), ursodeoxycholic acid reduced the proportion of Treg cells in tumor infiltrating lymphocytes.

### Example 3

Ursodeoxycholic acid inhibits tumor growth mainly by reducing the proportion of Treg cells in the microenvironment of tumors, while SHR-1210 (Hengrui Medicine), as a humanized anti-PD-1 antibody, blocks the binding between PD-1 and PD-L1 with its specificity, and terminates the PD-1 immunosuppression signals resulting from the interaction between PD-1 and PD-L1 in T cells.

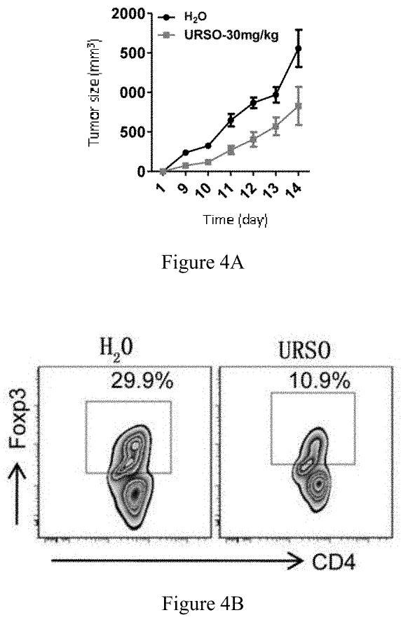

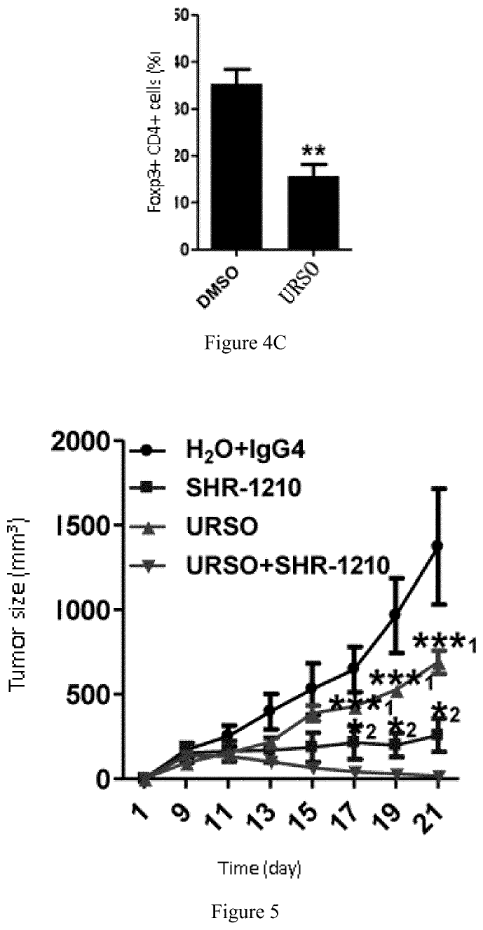

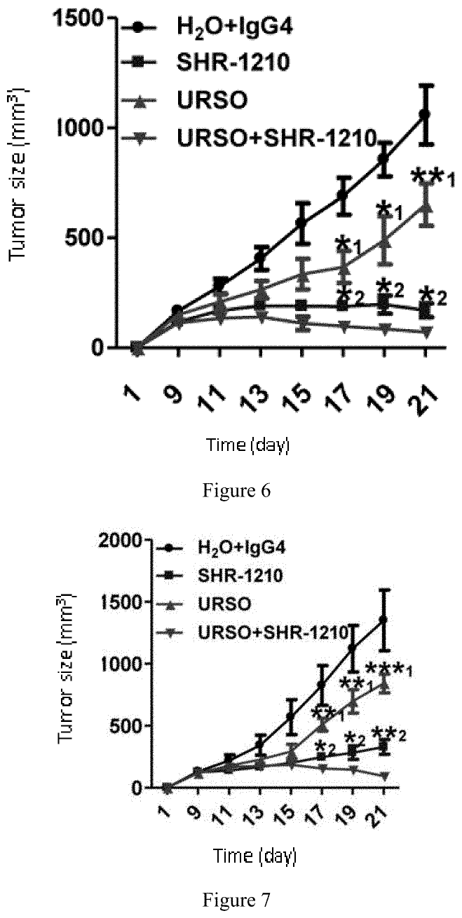

To verify whether the combined use of ursodeoxycholic acid and anti-PD-1 antibody can improve antitumor effect, colon cancer cell line MC38, melanoma cell line B16 and lung cancer cell line LLC are inoculated subcutaneously into humanized PD-1 transgenic mice, which were randomly divided into four groups when tumor volume reaches 100 mm³. Treatment groups were treated with SHR-1210, URSO or SHR-1210 in combination with URSO, while the control groups were injected with water and humanized IgG4 (an isotype control for SHRR-1210). SHR-1210 was used every two days at a dosage of 10 mg/kg, while URSO was used every other day at 30 mg/kg, with the volume of tumor recorded each time. According to the results shown in FIG. 5, FIG. 6 and FIG. 7, URSO and SHR-1210 significantly inhibited tumor growth, but combined therapy has obvious better therapeutic effect than monotherapy, wherein 1 represents comparison between URSO monotherapy and combined therapy using URSO and SHR-1210; 2 represents comparison between SHR-1210 monotherapy and combined therapy using URSO and SHR-1210; ***, ** and * represent P<0.001, P<0.01 and P<0.05 respectively.

### Example 4

To investigate the influence of ursodeoxycholic acid on humanized Treg cell differentiation, whole blood of volunteers were collected (ethics approval: approved by Medical Ethics Committee of Zhejiang University School of Medicine, Shen Lun No. 2019-013; five healthy volunteers, 10 ml peripheral venous blood each, collected by Zhejiang Medial & Health Group Hangzhou Hospital). naïve CD4+ T cells were sorted. Detailed procedures are as follows:

1. Separation of peripheral blood mononuclear cells:
(1) Fresh anticoagulant whole blood (EDTA, sodium citrate or heparin) was taken. Whole blood was diluted with isotonic solution (PBS or normal saline) of equal volume.
(2) A certain volume of separating medium (VESTA, #7111011) was added into a centrifuge tube, diluted blood sample was spread evenly on the surface of separating medium, and maintained a clear interface between two liquid surfaces. The volume ratio of separating medium, undiluted anticoagulant whole blood, and isotonic solution (PBS or normal saline) was 1:1:1.
(3) Under room temperature, the centrifuge tube was centrifuged at 700 g-800 g for 20-30 minutes in a horizontal rotor.
(4) After completion of centrifugation, at the bottom of the centrifuge tube were red blood cells, the intermediate layer was separating medium, the top-level layer was the plasma/tissue homogenate layer, and between the plasma layer and the separating medium layer was a thin layer of dense albugineous coat, i.e., mononuclear cell (including lymphocyte and monocyte) layer. The tunica albuginea layer was carefully suctioned into another centrifuge tube.
(5) The tunica albuginea layer in the other centrifuge tube was diluted with isotonic solution (PBS, normal saline or medium) to a certain volume, mixed well upside down, centrifuged at 250 g for 10 minutes in a horizontal rotor, and the culture supernatant was discarded. The content in the other centrifuge tube was washed for 1-2 times.
(6) Cells were resuspended with isotonic solution (PBS, normal saline or medium) for later use.

2. Sorting of human naïve CD4+ T cells:
(1) With Human naïve CD4+ T Cell Kit (STEMCELL, #19155) and the standard kit method adopted, naïve CD4+ T cells were sorted from human peripheral blood mononuclear cells, and plating was conducted according to the method in Example 1.
(2) Human T-Activator CD3/CD28 was added for T Cell Expansion and Activation (Gibco, #11161D).
(3) On day 4, Treg cell differentiation was detected by flow cytometry.

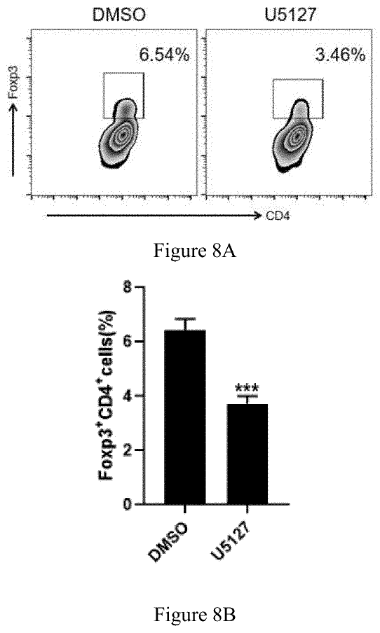

As shown in FIG. 8, the results indicated ursodeoxycholic acid reduced the differentiation of humanized endogenous Treg cells.

1. A method of preparing an antitumor drug comprising the step of utilizing ursodeoxycholic acid or its pharmaceutical salt.

2. The method according to claim 1 is characterized by: the ursodeoxycholic acid or its pharmaceutical salt functions by reducing the proportion of regulatory T cells in tumor infiltrating lymphocytes.

3. The method according to claim 1 is characterized by: that the ursodeoxycholic acid or its pharmaceutical salt is used in combination with an anti-PD-1 drug.

4. The method according to claim 1 is characterized by: the cancer targeted by the antitumor drug includes melanoma, rectal cancer and lung cancer.

5. An antitumor drug comprising ursodeoxycholic acid or its pharmaceutical salt in its content.

6. The antitumor drug according to claim 5 further comprising an anti-PD-1 drug.

7. The antitumor drug according to claim 6 wherein the anti-PD-1 drug is an anti-PD-1 monoclonal drug.

8. The antitumor drug according to claim 5 wherein the cancer targeted by the antitumor drug includes melanoma, rectal cancer and lung cancer.

9. A method of treatment of cancer comprising administering to a patient a pharmaceutically effective amount of ursodeoxycholic acid or its pharmaceutical salt.

10. The method according to claim 9 wherein the ursodeoxycholic acid or its pharmaceutical salt is used in combination with an anti-PD-1 drug.

11. The method according to claim 9, wherein the cancer targeted by the antitumor drug includes melanoma, rectal cancer and lung cancer.
